(19) United States
(12) Patent Application Publication
(10) Pub. No.: US 2024/0092879 A1
(43) Pub. Date: Mar. 21, 2024

(54) COMPOSITION OF HUMAN RECOMBINANT ANTIBODY FRAGMENTS THAT COMPLETELY NEUTRALIZE THE VENOM OF THE SCORPION CENTRUROIDES SCULPTURATUS

(71) Applicant: UNIVERSIDAD NACIONAL AUTONOMA DE MEXICO, CIUDAD DE MEXICO (MX)

(72) Inventors: Lidia RIAÑO UMBARILA, Morelos (MX); José Alberto ROMERO MORENO, Morelos (MX); Lourival Domingos POSSANI POSTAY, Morelos (MX); Baltazar BECERRIL LUJÁN, Morelos (MX)

(73) Assignee: UNIVERSIDAD NACIONAL AUTONOMA DE MEXICO, CIUDAD DE MEXICO (MX)

(21) Appl. No.: 17/937,666

(22) Filed: Oct. 3, 2022

(30) Foreign Application Priority Data
Sep. 20, 2022 (MX) A/2022/011698

(51) Int. Cl. C07K 16/18; A61P 39/00

(52) U.S. Cl. CPC C07K 16/18; A61P 39/00; A61K 2039/505; C07K 2317/21; C07K 2317/55; C07K 2317/622

(57) ABSTRACT

The present invention relates to a new composition of human recombinant antibody fragments in both single chain or Fab format, which is capable of completely neutralizing the venom of the scorpion Centruroides sculpturatus Ewing (C. sculpturatus. In particular the new composition comprises the scFv fragment 10FG2 (SEQ. ID. No: 1), which has broad cross-reactivity against various Mexican scorpion venom toxins, and the scFv fragment LR (SEQ. ID. No: 2), with more limited cross-reactivity. Alternatively, the new composition comprises the Fab 10FG2 fragment (SEQ. ID. No: 3 and SEQ.ID. No:4) and the Fab LR fragment (SEQ. ID. No: 5 and SEQ.ID. No 6). The two antibody fragments recognize independent epitopes, present in the two main toxins of the scorpion C. sculpturatus, so that they do not interfere with each other during their binding to the same, on the contrary the antibody fragments of the present invention complement the neutralizing activity.

### REFERENCE TO AN ELECTRONIC SEQUENCE LISTING

The contents of the electronic sequence listing (sequence-listing.xml; Date of Creation: Oct. 28, 2022; and Size: 12,395 bytes) is herein incorporated by reference in its entirety.

### BACKGROUND

The Centruroides sculpturatus Ewing scorpion (C. sculpturatus) is one of the toxic species of North America which is distributed in the United States (Arizona, California (southeastern border), Nevada (southern border), New Mexico (western border) and Utah) and on the border with the Mexican state of Sonora. (Gonzalez-Santillan and Possani 2018). In the United States it is considered responsible for the majority of envenoming cases (Kang and Brooks 2017), with an estimated incidence of approximately 9,000 cases per year (Gummin, Mowry et al. 2020). Regarding toxicity, it is the least toxic of the Centruroides species evaluated so far with an LD₅₀ of 22.7 μg/20 g of mouse. (Riano-Umbarila, Rodriguez-Rodriguez et al. 2017). The recent characterization of the venom showed the presence of two main toxic components, CsEM1a and CsEd that correspond to 8% and 1.6% in the venom respectively. (Carcamo-Noriega, Olamendi-Portugal et al. 2018). Like other Mexican scorpion toxins, these also modify the activity of mammalian sodium channels. (Schiavon, Pedraza-Escalona et al. 2012, Olamendi-Portugal, Restano-Cassulini et al. 2017, Gomez-Ramirez, Riano-Umbarila et al. 2020).

### SUMMARY

### Brief Summary

Although a commercial antivenom of equine origin is currently available (Boyer, Degan et al. 2013), the alternative of producing one on antibody fragments of human origin is novel, with the advantage of avoiding the arduous collection and sacrifice of thousands of scorpions, as well as the use of horses and the dependence on their heterogeneous immune response.

The present disclosure relates to a new composition of human recombinant antibody fragments in either the single chain or Fab formats. In particular a new composition of two single chain antibody variant fragments (scFv) or Fab is disclosed. In particular the new composition comprises the antibody fragment 10FG2, either in its scFv format (SEQ. ID. NO: 1) or in its Fab format (SEQ. ID. NO: 3 and SEQ. ID. NO:4), which have broad cross-reactivity against various Mexican scorpion venom toxins, and the antibody fragment LR, either in its scFv format (SEQ. ID. NO: 2) or in its Fab format (SEQ. ID. NO: 5 and SEQ. ID. NO: 6) with more limited cross-reactivity. The new composition is characterized by the fact that the two antibody fragments that form it, recognize the toxins of the scorpion Centruroides sculpturatus Ewing (C. sculpturatus), through two independent epitopes, so they do not interfere with each other during their binding to them. A mixture of these two fragments shows a complementary neutralizing activity, since to neutralize the venom, lower concentrations of these fragments are required, compared to that necessary individually to achieve a neutralization of at least 90% of the population. On the other hand, from a therapeutic point of view, neutralization by means of the composition of the present disclosure leaves no symptoms of envenoming, compared to neutralization achieved with either of the two antibody fragments separately. The two scFv fragments of the new composition showed affinities between 1.8×10⁻⁹ and 6.1×10⁻¹⁰ M. The scFv 10FG2 showed a higher protective capacity as it was able to neutralize up to 5 DL₅₀ of C. sculpturatus venom using a molar ratio of 1:2 (toxin:scFv), although with some remaining symptoms of intoxication in the treated mice. This disclosure, particularly comprises the use of both scFvs to elaborate a composition that, at molar ratios as low as 1:5:5 (toxins: scFv 10FG2: scFv LR), was able to rescue animals intoxicated with 3 DL₅₀ of the venom of C. sculpturatus, recovering the normal behavior of the treated mice. Therefore, the new composition comprises two antibody fragments either in scFvs (SEQ. ID. NO: 1 and SEQ. ID. NO: 2) or Fab (SEQ. ID. NO: 3 and SEQ. ID. NO:4 and SEQ. ID. NO: 5 and SEQ. ID. NO:6) format with complementary activities during neutralization of C. sculpturatus venom.

We currently have two neutralizing antibody fragments, scFvs LR and 10FG2, derived from the parental scFvs 3F and C1 respectively, isolated by phage display processes from a non-immune human scFvs library and using as target the Cn2 toxin from the venom of the scorpion Centruroides noxius (Riano-Umbarila, Juarez-Gonzalez et al. 2005). The scFvs 3F and C1, were subjected to several processes of directed evolution to increase on the one hand the affinity towards the Cn2 toxin as well as the recognition towards other Mexican scorpion venom toxins such as: Css2 (from C. suffusus) CII1 and CII2 (from C. limpidus) and Ctla (from C. tecomanus). Thus, from scFv 3F, scFv LR was generated, which is able to neutralize the Cn2 and Css2 toxins, as well as the corresponding whole venoms. (Riano-Umbarila, Contreras-Ferrat et al. 2011). Similarly, scFv 10FG2 was generated, which neutralizes Cn2, Css2, CII1, CII2, Ct1a, Cell9 toxins (from C. elegans), as well as the venoms of C. noxius, C. suffusus, C. infamatus, C. hirsutipalpus and C. spp nov. from Cumpas Sonora, Mexico. (Riano- Umbarila, Gomez-Ramirez et al. 2019). This broad cross-neutralization of scFv 10FG2 is explained by the high sequence identity of the toxins, the conservation of the disulfide bridge pattern and 3D structures. (Lopez-Giraldo, Olamendi-Portugal et al. 2020). On the other hand, these scFvs have been extensively characterized and we know that they are monomeric proteins with Tms close to 60° C. (Riano-Umbarila, Rojas-Trejo et al. 2020) and, due to their size, they are rapidly distributed in the organism, which is a great advantage in cases of acute intoxication by scorpion sting.

Taking into account the above, it is necessary to continue evaluating the neutralization capacity of these scFvs against other venoms of medical importance, due to their potential use as a new generation antivenom against several species of Mexican and North American scorpions.

### Summary of the Present Disclosure

The present disclosure relates to a new composition of human recombinant antibody fragments in both single chain and Fab format, which is capable of completely neutralizing the venom of the scorpion Centruroides sculpturatus Ewing (C. sculpturatus). In particular there is disclosed a new composition of two antibody variant fragments which may be in either single chain (scFv) or Fab format. In particular the new composition comprises the scFv fragment 10FG2 (SEQ. ID. No: 1), which has broad cross-reactivity against various Mexican scorpion venom toxins, and the scFv fragment LR (SEQ. ID. No: 2), with more limited cross-reactivity. Alternatively, the new composition comprises the Fab 10FG2 fragment (SEQ. ID. No: 3 and SEQ.ID. No:4) and the Fab LR fragment (SEQ. ID. No: 5 and SEQ.ID. No 6). The new composition is characterized in that the two antibody fragments it comprises recognize independent epitopes, present in the two main toxins of the scorpion toxin C. sculpturatus, so that they do not interfere with each other during their binding to the same, on the contrary the composition of the present disclosure complement the neutralizing activity since lower concentrations of both fragments are required to neutralize the venom, as compared to the concentrations required for either of them separately, besides eliminating the residual side effects that either of them allows, after neutralization. The two scFv fragments of the new composition showed affinities between 1.8×10⁻⁹ and 6.1×10⁻¹⁰ M. Individually, the scFv 10FG2 showed a better protective capacity in mice as it was able to neutralize up to 5 DL₅₀ of C. sculpturatus venom, but with symptoms of intoxication. This disclosure particularly comprises the use of both scFvs in low molar ratios 1:5:5 (toxins: scFv 10FG2: scFv LR), to inhibit the toxic effect of the venom of C. sculpturatus, without showing symptoms of intoxication. Therefore, the new disclosure comprises the use of the two scFvs 10FG2 and LR (SEQ. ID. NO: 1 and SEQ. ID. NO: 2, respectively) or else of the two Fab fragments 10FG2 and LR, which are complementary during the neutralization of the venom of C. sculpturatus, for the preparation of a new composition capable of completely neutralizing the venom of C. sculpturatus.

### BRIEF DESCRIPTION OF THE FIGURES

FIG. 1. Image showing the alignment of the sequences of the toxins neutralized by scFvs LR (SEQ. ID. NO: 2) and 10FG2 (SEQ. ID. NO: 1) and the main toxins of C. sculpturatus venom (CsEd and CsEM1a in bold). Cn2, toxin 2 from C. noxius; Css2 and Css4, toxins 2 and 4 from C. suffusus; CsEd and CsEM1a, toxins from C. sculpturatus; Ct1a, toxin 1 from C. tecomanus; Cell9, toxin 2 from C. elegans; CII1 and CII2, toxins 1 and 2 from C. limpidus. a, neutralizing scFvs (Riano-Umbarila, Contreras-Ferrat et al. 2011, Riano-Umbarila, Gomez-Ramirez et al. 2019). Dots correspond to residues conserved with respect to toxin Cn2. Sequence identity of the CsEd and CsEM1a toxins to each other is between 86% and 92%.

FIG. 2A. Sensorgrams of the interaction between the toxins CsEM1a and CsEd with the scFvs LR (SEQ. ID. NO: 2) and 10FG2 (SEQ. ID. NO: 1) at the described concentrations, a 25° C., in a continuous flow of 50 μL min−1.

FIG. 2B. Competition analysis of both scFvs for binding to the most abundant toxin CsEM1a in a flow of 20 μL min−1 at 25° C. The concentration of each scFv was 500 nM.

FIG. 3A. Overlaying of the structural complexes of LR-Cn2-RU1 (black), LR-CsEd-10FG2 (gray), and LR-CsEM1a-10FG2 (white) shows minimal differences among them. The toxins are identified by having a core structure consisting of one α-helix and three β-strands (β-sheet).

FIG. 3B. Details of the binding interface between scFv 10FG2 (SEQ. ID. NO: 1) and toxin CsEM1a are shown. The toxin is shown in black; the scFv is shown in white. Y9 of CsEM1a established a variety of interactions such as hydrogen bonds with the Y60 main chain of scFv 10FG2, hydrophobic contacts with Y59 and Y60, 25 and aromatic-aromatic interaction with Y59, as well as a cation-Pi interaction with K65. Besides, Y59 residue showed hydrogen bonds with Q32 and main chain S8. FIG. 3C. Similar details for the interactions between scFv 10FG2 (SEQ. ID. NO: 1) and CsEd toxin. The toxin is shown in black; the scFv is shown in white. The participation of residue Y59 of 10FG2 in the interaction with the residues of the toxin K8 and Q32 stands out.

FIG. 3D. Details of the scFv LR-CsEd complex showing interactions of residues N10 of the CsEd toxin with N31 of scFv LR and E15 of toxin with S52, A33, R228. The toxin is shown in black; the scFv is shown in white.

FIG. 3E. Details of the scFv LR-CsEM1a complex at the same interface shown in FIG. 3D; interaction only occurs between residue E15 of toxin CsEM1a and R288 of scFv LR. The toxin is shown in black; the scFv is shown in white.

FIG. 4. Biacore analysis of Fab 10FG2 and LR interactions with CsEM1a toxin. Flow rate 50 μl/min, temperature 25° C.

FIG. 5. Biacore analysis of Fab 10FG2 and LR interactions with CsEd toxin. All samples were tested at the same nM concentration. Flow rate 50 μl min⁻¹, at a temperature of 25° C.

### DETAILED DESCRIPTION

### Definitions

The term “antibody” is used in the broadest sense and covers in particular monoclonal antibodies (including complete monoclonal antibodies), polyclonal antibodies and antibody fragments.

The term “antibody fragment” comprises a portion of a native antibody, which generally contains a segment involved in antigen binding. Examples of antibody fragments include Fab, F(ab′)2, Fv fragments, diabodies, single chain antibodies (scFv) and nanobodies.

The term “scFv” or “single chain Fv” antibody fragment corresponds to the VH and VL domains of an antibody, in which these domains are integrated into a single polypeptide chain. The scFv format further comprises a connecting peptide (linker) between the VH and VL domains. For a review of the scFv format, see Pluckthun in: The Pharmacology of Monoclonal Antibodies, vol. 113, Rosenburg and Moore eds. Springer-Verlag, New York, pp. 269-315 (1994).

The term “diabodies” refers to antibody fragments with two antigen binding sites, which are formed through the binding of two scFvs, were the variable heavy chain domain (VH) of the first scFv binds to the variable light chain domain (VL) of the second scFv. The incorporation of a short linker (10 amino acids or less) forces the formation of bimolecular structures where the VH and VL domains interact intermolecularly, thus generating two antigen binding sites. The diabodies are described more fully in, for example, EP 404,097; WO 93/11161; and Hollinger et al., Proc. Natl. Acad. Sci. USA 90: 6444-6448 (1993).

A “parental antibody” is an antibody or antibody-derived fragment comprising a particular amino acid sequence that is used as a starting point for directed evolution processes that including site-specific or random mutagenesis procedures (see Neylon, C., 2004. Nucleic Acids Research 32, 1448-148-1408), gene shuffling, among others; to generate antibody variants in one or more cycles of mutagenesis that modify said parental antibody by including one or more amino acid alterations in or adjacent to one or more hypervariable regions. The parental antibody may comprise a native (naturally occurring) sequence, including naturally occurring allelic variants or an antibody with pre-existing modifications in its amino acid sequence (insertions, deletions and/or substitutions). Preferably, the parental antibody would be of human origin.

As used herein, “antibody variant ” refers to an antibody generated by mutagenesis from a parental antibody of the present disclosure and possessing an amino acid sequence different from the amino acid sequence of said parental antibody. Preferably, the antibody variant comprises a heavy chain variable domain and a light chain variable domain with an amino acid sequence not present in nature. Such variants necessarily possess less than 100% identity or similarity to the amino acid sequence of the parental antibody. The antibody variants of the present disclosure bind with high affinity to the toxins of the venom of the scorpion C. sculpturatus.

“Amino acid alteration” refers to a change in the amino acid sequence resulting from the above-mentioned mutagenesis processes. Examples of alterations include insertions, deletions and in particular substitutions. To refer to a substitution, the amino acid present in the parental antibody is given followed by a number corresponding to the position it occupies in the parental antibody (starting at the amino end) and followed by the amino acid that replaces it, now present in the antibody variant.

“Neutralize, neutralization, or a neutralizing antibody”. Refers to the ability of the antibodies of the present disclosure to bind with high affinity to the CsEd and/or CsEM1a toxins, so as to mitigate their lethal effect when administered to a susceptible mammal, either in purified form or as part of the total venom of the corresponding scorpion species.

“Treatment” refers to therapeutic treatment.

The term “those in need of treatment” includes individuals who have already been stung by the C. sculpturatus scorpion.

“Isolated” nucleic acid molecule refers to a nucleic acid molecule identified and separated from other nucleic acid molecules present in the natural source of nucleic acids that code for an antibody, i.e. it is found outside its natural context. A mutant nucleic acid molecule is different in form or arrangement from that found in nature. Thus, isolated nucleic acid molecules are distinguished from nucleic acid molecules present in the cells that contain them. However, an isolated nucleic acid molecule is actually a molecule whose nucleotide sequence is present in the cells that ordinarily express the antibody, but this nucleic acid molecule, when cloned, is in a different context from the one it occupies in natural cells.

The term “effective amount” or “pharmacologically effective amount” of a compound in unit doses of the mixture depends on several factors. These factors include amounts of the other ingredients if used, and tolerance of the active ingredient of the compound. The effective amount of the active ingredient varies from about 0.1% to nearly 50% by weight, based on the total weight of the compound. For scorpions ting antivenoms, the F(ab′)2 type preparation contained in each vial is the amount necessary to neutralize from 135 to 220 half lethal doses.

“Pharmaceutically Acceptable Vehicle.” Refers to solid or liquid filler excipients, diluents, or substances that can be safely used in the systemic or topical administration of drugs. Depending on the particular route of administration, several vehicles well known in the industry are pharmaceutically acceptable and include solid or liquid fillers, diluents, hydrotropes, surfactants and encapsulating substances. The amount of vehicle used in conjunction with the F(ab′)2 fragments provide a manageable amount of material per unit dose of antivenom.

Pharmaceutically acceptable vehicles for systemic administration which may be incorporated in the composition of the disclosure include sugar, starches, cellulose, vegetable oils, buffers, polyols and alginic acid. Specific pharmaceutically acceptable vehicles are described in the following documents, all mentioned herein by reference: U.S. Pat. No. 4,401,663, Buckwalter et al., issued Aug. 30, 1983; European Patent Application No. 089710, LaHann et al., issued Sept. 28, 1983; and European Patent Application No. 0068592, Buckwalter et al., issued Jan. 5, 1983. Preferred vehicles for parenteral administration include propylene glycol, pyrrolidone, ethyl oleate, aqueous ethanol and combinations thereof. Representative vehicles include acacia, agar, alginates, hydroxyalkylcellulose, hydroxypropyl methylcellulose, carboxymethylcellulose, sodium carboxymethylcellulose, carrageenan, cellulose powder, guar gum (guaran), cholesterol, gelatin, agar gum, gum arabic, gum karaya, gum ghatti, locust bean gum, octoxynol 9, oleyl alcohol, pectin, polyacrylic acid and its homologues, polyethylene glycol, polyvinyl alcohol, polyacrylamide, sodium lauryl sulfate, polyethylene oxide, polyvinylpyrrolidone, glycol monostearate, propylene glycol monostearate, xanthan gum, tragacanth, sorbitan esters, stearyl alcohol, starch and its modifications. Suitable ranges vary from 0.5% to 1%.

The abbreviations most commonly used in the present disclosure and their meaning are: scFv (single chain antibody fragment variants); CDR (Complementarity Determining Region); FW (Framework Region); C. sculpturatus (Centruroides sculpturatus) and aa (amino acids).

“Complete Neutralization or Complementary Neutralization”. In the context of the present disclosure, it refers to the neutralization of the toxins present in the total scorpion venom by a composition comprising two or more scFvs, in which, using a complementary neutralization effect between the scFvs comprising the composition, the laboratory animals (mice) are not only kept alive, but the symptoms of intoxication gradually disappear over time after the application of the mixture thereof.

### DETAILED DESCRIPTION

Due to the abundance of scorpion species toxic to humans in North America, the optimal neutralization of venoms turns out to be a major challenge. For example, in Mexico a recent estimate proposes the existence of at least 21 toxic species in the country, although it cannot be discarded that eventually others could be identified. That is why the present disclosure takes advantage of new strategies to obtain a specific antivenom against scorpion stings, such as directed evolution and phage display, as well as of the cross-reactivity of antibodies and their fragments to achieve the neutralization of other toxins that share a high degree of identity (see example 1).

Particularly in the present disclosure we found that the main toxins of C. sculpturatus venom: CsEM1a and CsEd, have conserved epitopes with respect to the Cn2 and Css2 toxins, as determined in the present disclosure, which favors the binding of the scFvs 10FG2 (SEQ. ID. NO: 1) and scFvs LR (SEQ. ID. NO: 2) to said toxins. Determination of the interaction kinetic constants, allowed to observe that all the association constants (kₒₙ) are in the order of 10⁵ M⁻¹ s⁻¹, which manifests as a fast binding between the scFvs of the present disclosure and the toxins. In the case of the dissociation constant (k_{off}), from its values we were able to determine, in the present disclosure, that the retention times (R_{T}), show greater differences in the kinetic dissociation constant of the toxin-antibody complex. For example, we observed that the interaction of the scFv LR (SEQ. ID. NO: 2) remains for almost 71 min with the CsEd toxin (Table 1), which is the least abundant toxin in the venom, while with the CsEM1a toxin it was 21 min. The authors of the present disclosure have reported that, in order to achieve a good neutralization, in this case of Cn2 and Css2 toxins, the retention times must be longer than 25 min (Riano-Umbarila, Juárez González, et. al.2005). As CsEM1a is the most abundant toxin within the venom of C. sculpturatus and with the result of a shorter toxin-antibody retention time, it could be explained why the scFv LR (SEQ. ID. NO: 2) in the preliminary protection assays was not as efficient as the scFv 10FG2 (SEQ. ID. NO: 1) (see example 2). The latter recognized a different epitope, which requires shorter retention times to be neutralized (Riano-Umbarila, Gomez-Ramirez et al. 2019). That is why retention times of 49 and 63 min were sufficient to achieve inhibition of the toxic effect of the two toxins as indicated by 100% survival of mice injected with the complete venom (Table 2).

On the other hand, in the neutralization tests of the present disclosure we managed to demonstrate that scFv 10FG2 (SEQ. ID. NO: 1) is able to neutralize up to 5 LD₅₀ of venom, even using low molar ratios of 1:2 (toxin:scFv) (Table 3A), albeit with slight signs of intoxication in mice. These signs were completely eliminated when we used the new composition comprising the scFvs 10FG2 (SEQ. ID. NO: 1) and LR (SEQ. ID. NO: 2). Importantly, when the two scFvs interact simultaneously with a toxin, they are able to coat about 75% of its surface. This toxin coating effect is similar to what would be occurring with polyclonal antivenoms produced in horses. In the case of the scFvs of the present disclosure, this coating is achieved with only two scFvs, with a mass close to 25% of the mass of a (Fab′)2. This implies that the same effect is achieved with significantly less protein. With these promising results, in the present disclosure we decided to make a more demanding evaluation of the neutralizing capacity of the new composition, through a rescue assay (Knudsen, Casewell et al. 2020) (Table 3B). After causing for 10 min a strong intoxication with 3 DL₅₀ of venom, the new composition of both scFvs (LR and 10FG2 in equivalent amounts) was administered so that the mixture constituted a 1:5 molar ratio (toxin:scFv) of each one. While in the control group the death of the mice occurred between 30 min and 1 h after injection, in the group of rescued mice, the signs of intoxication decreased progressively until showing a completely normal behavior of the mice within 30 min. This is the first known rescue using this molar ratio (1:5:5). This result is important because it is a demanding evaluation, given that scFvs are not administered intravenously, where there would be a more rapid distribution of the antivenom.

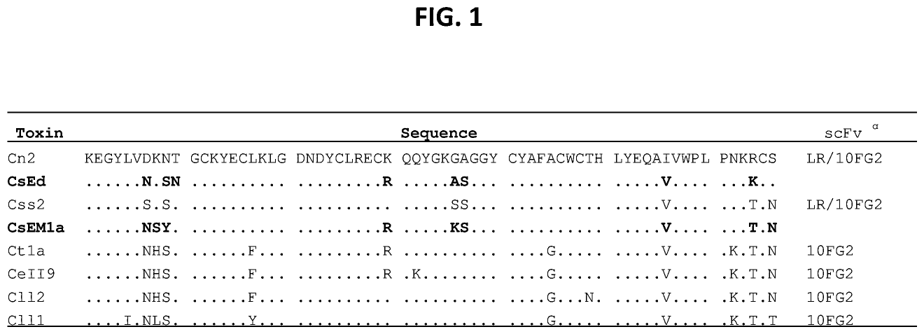

In the present disclosure the molecular dynamics (MD) analysis showed that all complexes studied here retain most of the interactions or contacts (Tables 4 and 5), by virtue of the similarities between the structures of the CsEM1a and CsEd toxins. The MDs of scFv 10FG2 (SEQ. ID. NO: 1) in complex with the toxins, explain why the binding with the CsEM1a toxin has a higher affinity compared to CsEd. Regarding the average number of contacts along the MDs, there is no remarkable difference between the complexes studied for both toxins in the present disclosure. The average number of hydrogen bonding contacts is 9.1 and 10.2, for CsEM1a and CsEd, respectively. However, their differences lie in other aspects. Between these two toxins there are only six differences between their amino acid sequences (see FIG. 1). Of these differences, only three were at the binding interface corresponding to the amino acids at positions 8, 9 and 10.

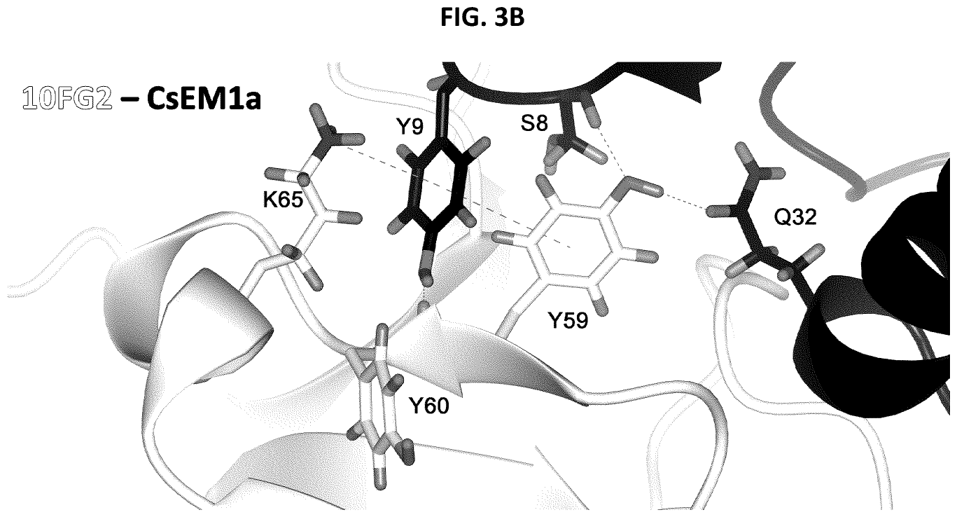

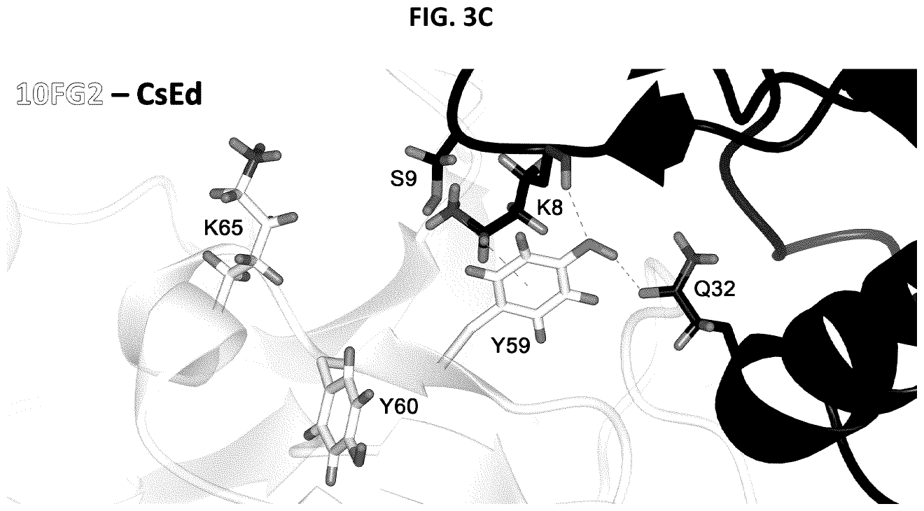

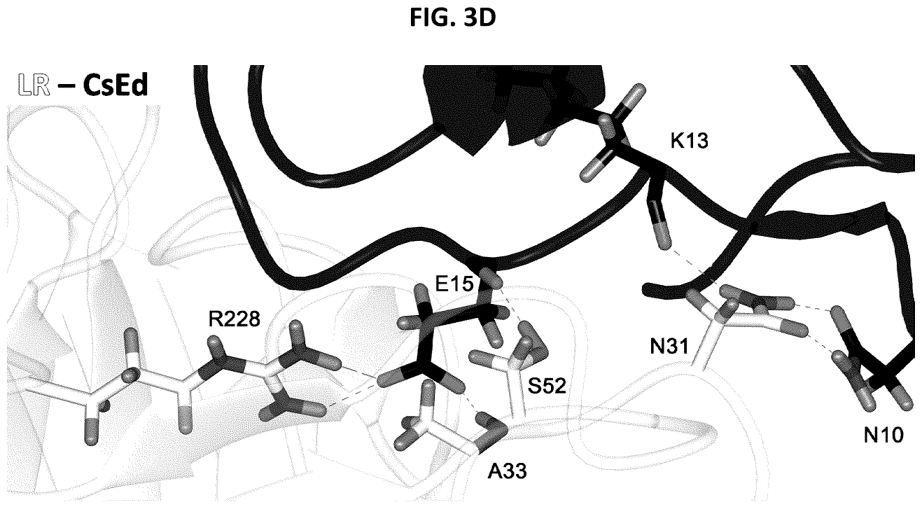

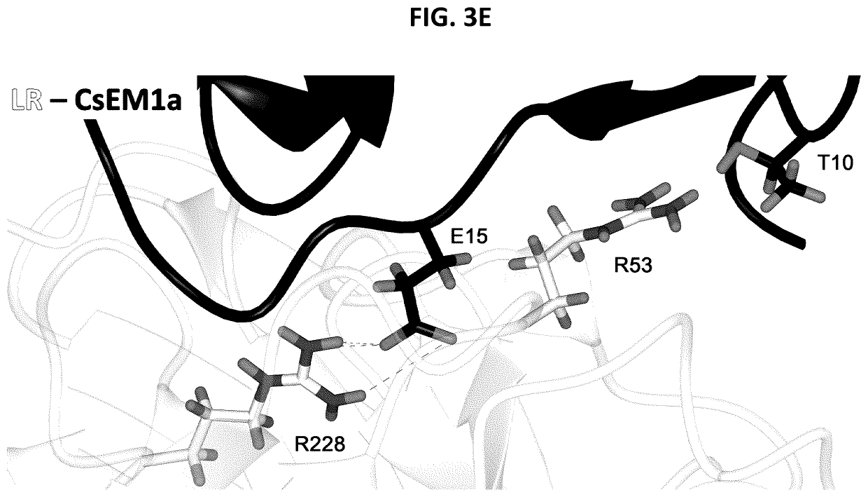

In FIGS. 3B and 3C it can be seen that Y9 of CsEM1a established a variety of interactions such as hydrogen bonds with the Y60 main chain of scFv 10FG2, hydrophobic contacts with Y59 and Y60, an aromatic-aromatic interaction with Y59, as well as a cation-Pi interaction with K65. This is in contrast to S9 of the CsEd toxin (Table 4). When analyzing the region of the structure where Y9 is located, it is observed that the temperature B-factors are lower for these three residues S8, Y9 and T10 (Table 6). This data suggested that the contacts established by Y9 stabilize the site, which contributes to the increased affinity of scFv 10FG2 (SEQ. ID. NO: 1) for the CsEM1a toxin (see Example 3).

On the other hand, in the present disclosure it was determined that in the case of the complexes composed of the scFv LR (SEQ. ID. NO: 2) and the toxins CsEd and CsEM1a, the average number of hydrogen bond contacts along the MD was 13.1 for the case of the CsEd toxin, whereas for the CsEM1a toxin it was 8.79. The scFv LR (SEQ. ID. NO: 2) recognized CsEd better, for which, the MD (Table 5) showed several hydrogen bonds contacts of the N31 of the scFv LR with residues E15, K13 and N10 (FIG. 3C), which do not occur with the CsEM1a toxin (FIG. 3D). Hydrogen bond could also be observed with CsEd toxin via E15 with A33 of scFv LR. Another residue of the CsEd toxin that interacts with the LR scFv (SEQ. ID. NO: 2) is S54, whereas for CsEM1a, it is R27. These sequence variations between the two toxins influenced the difference in their affinities for the scFvs where the affinity for CsEd was higher. It is important to note that these CsEd toxin residues, with the exception of N10, are found in CsEM1a toxin and Cn2 toxin. The affinity of scFv LR (SEQ. ID. NO: 2) is significantly higher for Cn2 toxin (KD=1.12×10⁻¹⁰ M, and R_{T}=333 min) (Riaño et al 2011)). This toxin, like CsEM1a, does not present contacts with N31 of scFv LR; however, Cn2 establishes salt-bridge type contacts through its D7 with R53 of scFv (see Table 5). These contacts were not observed with the CsEd or CsEM1a toxins. Salt bridges have a higher energy than hydrogen bonds in general, which could explain the difference in affinity for the Cn2 toxin with respect to the other two toxins studied. The observation that the same residues present in the three toxins do not make the same contacts with the scFvs LR (SEQ. ID. NO: 2) or 10FG2 (SEQ. ID. NO: 1), suggests that there are additional factors contributing to the observed differences in affinities and in the MDs themselves. One possible explanation is related to the small differences at the sequence level (e.g. there is 91% identity between CsEd and CsEM1a, and 88% identity between Cn2 and CsEd) that may determine differences in matching of scFv and toxins at the MD level.

The scFvs LR (SEQ. ID. NO: 2) and 10FG2 (SEQ. ID. NO: 1) whose basic characteristics have been previously disclosed, are employed in the present disclosure as elements to elaborate a new composition for an antivenom against the sting of the scorpion C. sculpturatus in Mexico, United States of America and any other country where it is found. In the present disclosure it is reported for the first time that the scFv 10FG2 (SEQ. ID. NO: 1) neutralizes the effect of the venom of C. sculpturatus, although it still leaves slight signs of intoxication in mice and although the scFv LR (SEQ. ID. NO: 2) does not neutralize it totally, the composition comprising the combination of both scFvs, have a complementary effect. The mixture of these scFvs, improves the efficiency with which the venom is neutralized and above all the residual symptoms of envenoming in mice are eliminated, i.e. a complete neutralization is achieved. The study of the molecular interactions between the scFvs and the toxins, revealed in the present disclosure that many of the relevant contacts are maintained, so it was not necessary to perform affinity optimization processes towards any of the toxins of C. sculpturatus.

TABLE 1
Kinetic constants of the interaction of scFvs and toxins

| | | kon (M⁻¹ | koff | | |
|---|---|---|---|---|---|
| | | s⁻¹) × | (s⁻¹) × | K_{D} | TR |
| scFv | Toxin | 10⁵ | 10⁻⁴ | (M) | (min) |
| LR (SEQ. ID. | CsEd | 3.85 | 2.35 | 6.1 × 10⁻¹⁰ | 70.9 |
| NO: 2) | CsEM1a | 6.28 | 7.9 | 1.29 × 10⁻⁹ | 21 |
| 10FG2 (SEQ. | CsEd | 1.85 | 3.4 | 1.84 × 10⁻⁹ | 49 |
| ID. NO: 1) | CsEM1a | 2.37 | 2.61 | 1.1 × 10⁻⁹ | 63 |

Biacore assays were performed at 25° C. using a flow rate of 50 μL min⁻¹. The affinity constants (K_{D}) were calculated using the Langmuir model (1:1) in BIAevaluation 3.1 software. R_{T}: residence time.

In another embodiment of the present disclosure, in order to illustrate but not limit, the possibility of changing antibody fragment format. Fab version or format were generated from the scFv 10FG2 and LR.

It has been much discussed that the scFv antibody format may be more unstable than Fab, since in the latter there is a larger interface generated by the domains of the VL- CL regions with the VH-CH domains (Rothlisberger D, et al. 2005) compared to that of the VH and VL domains of scFv.

Therefore, the sequences of the heavy and light variable domains of the scFv LR and 10FG2 were used for the constructs of the corresponding heavy and light chain genes of the corresponding Fab: (VL-CL) and (VH-CH). The genes were synthesized by Genscript company and expressed in E coli, using the same protocol as for the scFvs.

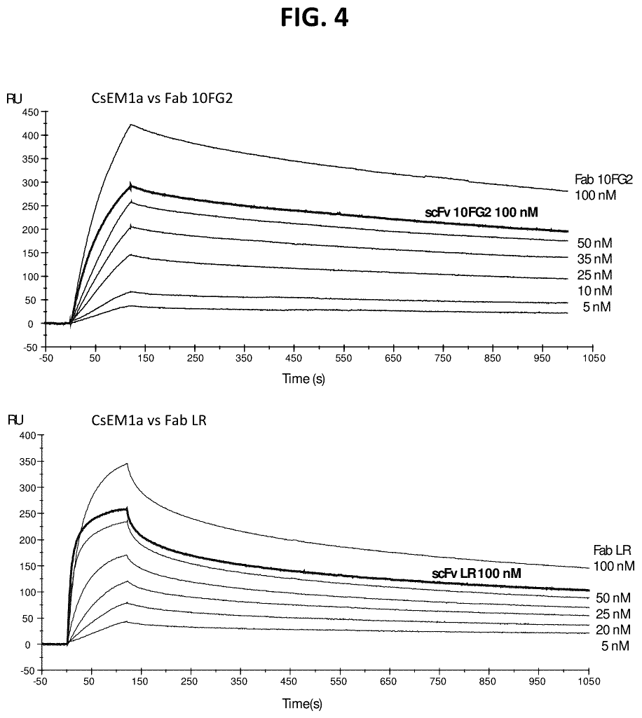

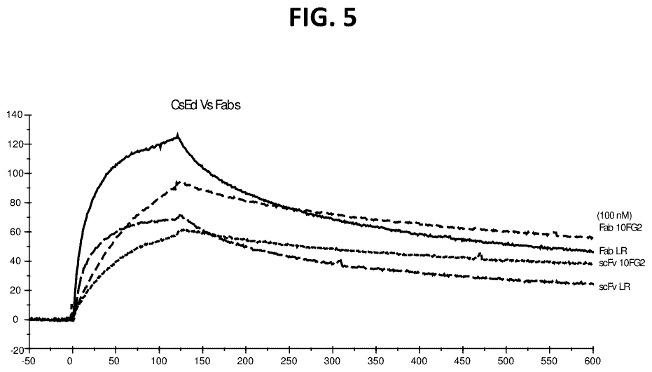

Biacore recognition analyses of Fab LR and Fab 10FG2 against the toxins confirmed that the interaction is similar to the corresponding scFvs in terms of the profile of the curves. The only difference is due to the level of the response, since the mass of the Fab is about 50 KDa, while that of the scFv is about 28 KDa, so the response, in terms of resonance units, is higher in the Fab, at the same concentration. However, the curve is similar, that is to say that the change of format did not alter the recognition properties as can be observed in the sensorgrams against the most abundant toxin of C. sculpturatus CsEM1a. Where we see that its behavior, at different molar concentrations, is similar to the curve of the scFvs 10FG2 and LR (see FIG. 4). The interaction with the CsEd toxin at a concentration of 100 nM of both scFvs and Fabs was also analyzed and the behavior is similar as described in the previous case (FIG. 5).

For this reason, Fabs are equivalent in recognition to their corresponding scFvs, so either format can contribute to the neutralization of C. sculpturatus venom.

Thus, the C. sculpturatus scorpion venom neutralizing composition of the present disclosure comprises a 10FG2 antibody fragment, either in its scFv format (SEQ. ID. ID. NO: 1) or in its Fab format (SEQ. ID. NO:3 and SEQ. ID. NO:4) and an LR antibody fragment, either in its scFv format (SEQ. ID. NO: 2) or in its Fab format (SEQ. ID. NO:5 and SEQ. ID. NO:6).

One embodiment of the disclosure is directed to a composition that neutralizes Centruroides sculpturatus scorpion venom, comprising an antibody fragment 10FG2, and an antibody fragment LR. In the composition the antibody fragments 10FG2, and LR, may be in scFv format, having sequences SEQ. ID. NO: 1 and SEQ.ID. NO:2, respectively. In the composition, the antibody fragments 10FG2, and LR, may be in Fab format, having amino acidic sequences SEQ. ID. NO: 3 for the light chain and SEQ.ID. No:4 for the heavy chain, as well as SEQ. ID. NO: 5 for the light chain and SEQ.ID. NO:6 for the heavy chain, respectively. In the composition, the composition may completely neutralize Centruroides sculpturatus scorpion venom.

One embodiment of the disclosure is directed to a pharmaceutical composition that neutralizes Centruroides sculpturatus scorpion venom, comprising an antibody fragment 10FG2, and an antibody fragment LR. and a pharmaceutically acceptable vehicle. In the pharmaceutical composition, the antibody fragments 10FG2, and LR, may be in scFv format, having amino acidic sequences SEQ. ID. NO: 1 and SEQ. ID. NO: 2, respectively. In the pharmaceutical composition, the antibody fragments 10FG2, and LR, may be in Fab format, having amino acidic sequences SEQ. ID. NO: 3 for the light chain and SEQ.ID. No:4 for the heavy chain, as well as SEQ. ID. NO: 5 for the light chain and SEQ.ID. NO:6 for the heavy chain, respectively. The pharmaceutical composition of this disclosure may completely neutralize Centruroides sculpturatus scorpion venom.

One embodiment of the disclosure is directed to a method for treating Centruroides sculpturatus scorpion envenoming comprising the step of administering a pharmaceutical composition of this disclosure to neutralize the

Centruroides sculpturatus scorpion venom. In one aspect of this embodiment, the pharmaceutical composition may comprise an antibody fragment 10FG2, and an antibody fragment LR, and optionally a pharmaceutically acceptable vehicle. In a second aspect of this embodiment, the pharmaceutical composition may comprise antibody fragments 10FG2, and LR, in scFv format, having amino acidic sequences SEQ. ID. NO: 1 and SEQ. ID. NO: 2, respectively; and optionally a pharmaceutically acceptable vehicle. In a third aspect of this embodiment, the pharmaceutical composition may comprise antibody fragments 10FG2, and LR, in Fab format, having amino acidic sequences SEQ. ID. NO: 3 for the light chain and SEQ.ID. No:4 for the heavy chain, as well as SEQ. ID. NO: 5 for the light chain and SEQ.ID. NO:6 for the heavy chain, respectively; and optionally a pharmaceutically acceptable vehicle.

All compositions, methods, treatments, pharmaceutical compositions of this disclosure may be for the treatment of a mammal such as, for example, a human. The mammal may be afflicted with Centruroides sculpturatus scorpion envenoming. The compositions, methods, treatments, pharmaceutical compositions of this disclosure may completely neutralize Centruroides sculpturatus scorpion venom.

### Materials and Methods

### Venom and Toxins

For the present disclosure the venom of C. sculpturatus was purchased from Spider Pharm and venom company of Santa Rita Foothills Yarnell AZ. USA. The lyophilized venom was resuspended in tetra-distilled water and centrifuged at 14,000 rpm, for 15 min at 4° C. Insoluble material was discarded, and the supernatant containing the toxins was recovered and quantified in the spectrophotometer at a λ=280 nm. Forty mg of venom was fractionated and the toxins isolated following the procedure described in (Carcamo-Noriega, Olamendi-Portugal et al. 2018) to obtain CsEM1a and CsEd toxins.

Protein Expression of scFvs 10FG2 (SEQ. ID. NO: 1) and LR (SEQ. ID. NO: 2)

In the present disclosure protein expression and purification of each sequence was carried out using the pSyn1 plasmid and the E. coli TG1 strain, as described in Riano-Umbarila, Juarez-Gonzalez et al. 2005. The scFvs were always maintained in 1× PBS buffer (NaCl 137 mM, KCl 2.7 mM, Na₂ HPO₄ 8 mM, KH₂ PO_{4 1.5} mM, pH 7.4). Protein n was determined spectro-photometrically at λ=280 nm.

### Surface Plasmon Resonance Recognition and Affinity Determinations

In the present disclosure for recognition and affinity assays we used CM5 chips, the Amine Coupling Kit (Biacore) and a Biacore biosensor system (Biacore X, Uppsala, Sweden). For each toxin, 250 ng were dissolved in 100 μL of 10 mM 2-(N-morpholino) ethanesulfonic acid pH 6. Ten UL of toxin solution was bound to cell 2 of the previously activated CM5 sensor chip at a flow rate of 5 μL min⁻¹. Toxins were coupled at a density of approximately 100 resonance units (RU). After coupling, and during the assays, cell 1 (unbound) was used as a control. Protein solutions of the scFvs were diluted serially in HBS-EP buffer (Biacore); 100 μL of scFv samples were injected into each chip (CsEM1a or CsEd coupled) at a flow rate of 50 μL min⁻¹. Measurements were performed at 25° C. The scFv proteins were assayed at concentrations ranging from 0.5 nM to 180 nM. The dissociation phase lasted for 1000 s. The chip surfaces were regenerated with 10 mM Glycine-HCl pH=2. The kinetic constants were determined using the corresponding sensograms, which were corrected by subtracting the values of both the reference flow cell and the blank buffer injection. The Langmuir model (1:1) of the BIAevaluation software version 3.1 (Biacore, Uppsala, Sweden) was used for kinetic constants determination.

### Competition Assays by Surface Plasmon Resonance

In the present disclosure competition assays were performed by SPR to confirm that the scFvs bind to different epitopes of the toxins. The sensor chip was prepared as described above. Three saturating amounts (30 μL of 0.5 mM) of scFv 10FG2 (SEQ. ID. NO: 1) were consecutively injected onto CsEM1a-coated chip, the flow rate used was 20 μL min⁻¹ in HBS-EP buffer, until saturation of the available sites. Subsequently, 30 μL of scFv LR (SEQ. ID. NO: 2) was injected at a concentration of 0.5 mM. As control, a sample of scFv LR (SEQ. ID. NO: 2) recognizing CsEM1a was injected and the sensorgram was analyzed.

### Venom Neutralization Assays

### Mix Assay

In the present disclosure, to evaluate the neutralization activity against the whole venom, groups of 6 female CD1 mice were used in most cases (except in 2 cases groups of 10 animals were used). Samples were administered by intraperitoneal injection, following the protocols approved by the Bioethics Committee of the Biotechnology Institute of the UNAM, Mexico. In a preliminary trial, an amount of venom equivalent to 1 or 2 LD50 (23 μg or 46 μg venom) was mixed just with the scFv 10FG2 (SEQ. ID. NO: 1) or scFv LR (SEQ. ID. NO: 2) and the mixture scFvs 10FG2 (SEQ. ID. NO: 1) plus LR (SEQ. ID. NO: 2). The controls correspond to 1 or 2 DL50 of all venom in 1× PBS buffer to confirm venom activity. Subsequently, scFv(s) and venom mixing assays were performed at various toxin: scFv molar ratios. These ratios were calculated for two toxins present in the venom. The LD50 of C. sculpturatus venom is ˜23 μg/20 g mouse (Riano-Umbarila, Rodriguez-Rodriguez, et. al. 2017), where toxins represent 9.6˜10% of the venom. The amount corresponding to 2, 3, 4 or 5 LD50 of the venom was mixed with a fixed amount of scFv 10FG2 (SEQ. ID. NO: 1) or 10FG2 plus LR (SEQ. ID. NO: 2). The mixture of venom and scFv(s) was preincubated at room temperature (˜25° C.) for 30 min prior to injection into mice.

### Rescue Tests

In the present disclosure this experiment was performed to evaluate the ability of scFv 10FG2 (SEQ. ID. NO: 1) in combination with scFv LR (SEQ. ID. NO: 2), to rescue mice that were previously envenomed with 3 LD₅₀ (69 μg of venom). A time lag of 5 to 10 min was allowed to elapse before injecting mice with both scFv using toxin: scFv molar ratios of 1:5 of each. As described, the relative molar ratios were established assuming that 10% of the venom corresponds to toxic components.

Modeling and Structural Analysis of scFv 10FG2-CsEM1a, scFv 10FG2-CsEd, scFv LR-CsEM1a and scFv LR-CsEd Complexes

In the present disclosure with the aim of exploring the structural basis of the neutralization of CsEM1a and CsEd toxins by scFv 10FG2 (SEQ. ID. NO: 1) or scFv LR (SEQ. ID. NO: 2), models of these scFv in complex with these two toxins were prepared, based on the structural model of the ternary scFv RU1-Cn2-LR complex. (Riano-Umbarila, Ledezma-Candanoza et al. 2016). LR is one of the scFvs of the present disclosure and scFv RU1 is one of the predecessors of scFv 10FG2, both recognize and neutralize Cn2 toxin, thus allowing us to study the interactions withCsEd and CsEM1a toxins, by molecular replacement. In addition, we also used the model of the scFv 10FG2-Cn2 complex, previously generated (Riano-Umbarila, Gomez-Ramirez et al. 2019). Using the Maestro Program [Schrödinger Release 2021-1: Maestro, Schrödinger, LLC, New York, NY, 2021], the 10FG2-Cn2 complex was modified by replacing the amino acids necessary to transform the Cn2 toxin into the CsEM1a or CsEd toxins according to the amino acid sequences shown in FIG. 1. The three models (10FG2 (SEQ. ID. NO: 1) in complex with Cn2, CsEM1a or CsEd toxins, were adjusted with the Protein Preparation Wizard module provided with the Master Program and a 15 15 Å buffered box of water with 0.15 M NaCl was added using the System Builder module and adjusted to minimize volume. The models were subjected to energy minimization procedures until 0.1 Kcal/mol/A was reached and then adjusted to a minimum of 2000 repetitions, 3 LBFGS vectors and a minimum of 20 SD steps (steepest descent). The three scFv 10FG2-toxin complexes were subjected to molecular dynamics (MD) simulation procedures using the Desmond Program. (Kevin J. Bowers, Brent A. Gregersen et al. 2006). Using the Viparr utility provided with the Desmond program, the Charmm22star force field and the spatial water model force field were established in the three systems. They were then subjected to molecular dynamics (MD) simulation in the Desmond program with the following settings: an MD simulation time of 100 ns; trajectory log intervals of 10 ps (picoseconds) and five ps for energy logs; the NPT ensemble class was set to a temperature of 300 K. The Langevin thermostat and barostat methods were used to control temperature and pressure, with 100 ps relaxation time for both methods. We used an integration time step of two ps and a Coulombic cutoff radius of 9 Å (default value). A sample of twenty structural frames from each model complex was extracted at pairwise intervals from the trajectories generated by the MD simulations, for analysis of the interactions at the interface between scFv 10FG2 (SEQ. ID. NO: 1) and the different toxins. From this sample, one out of 5000 frames was taken and sent to the PIC (Protein Interactions Calculator) software using default values (Tina, Bhadra et al. 2007) and to the PISA software (Krissinel and Henrick 2007) software for analysis of the interface between scFv 10FG2 (SEQ. ID. NO: 1) and each of the toxins evaluated.

### EXAMPLES

The following examples are presented by way of illustrating some of the ways to obtain or use the present disclosure. It is possible to carry out many variations thereof without departing from the scope of the present disclosure and therefore should in no way be considered as limiting in any way the scope of the present disclosure.

### Example 1. Alignment of C. sculpturatus Venom Toxin Sequences With Toxins That are Neutralized by scFvs 10FG2 (SEQ. ID. NO: 1) and LR (SEQ. ID. NO: 2)

Due to the broad neutralizing capacity of scFv 10FG2 (SEQ. ID. NO: 1) (Riano-Umbarila, Gomez-Ramirez et al. 2019) and the good affinity of scFv LR (SEQ. ID. NO: 2) for some toxins, in the present disclosure it was decided to use scFv 10FG2 (SEQ. ID. NO: 2) as a neutralizing agent (Riano-Umbarila, Contreras-Ferrat et al. 2011). In the present disclosure it was decided to evaluate the neutralizing capacity of these antibody fragments on other venoms of scorpion venoms of the genus Centruroides. Initially, an alignment of the sequences of the toxins of the venom of C. sculpturatus with the toxins that are neutralized by these scFvs was performed (FIG. 1).

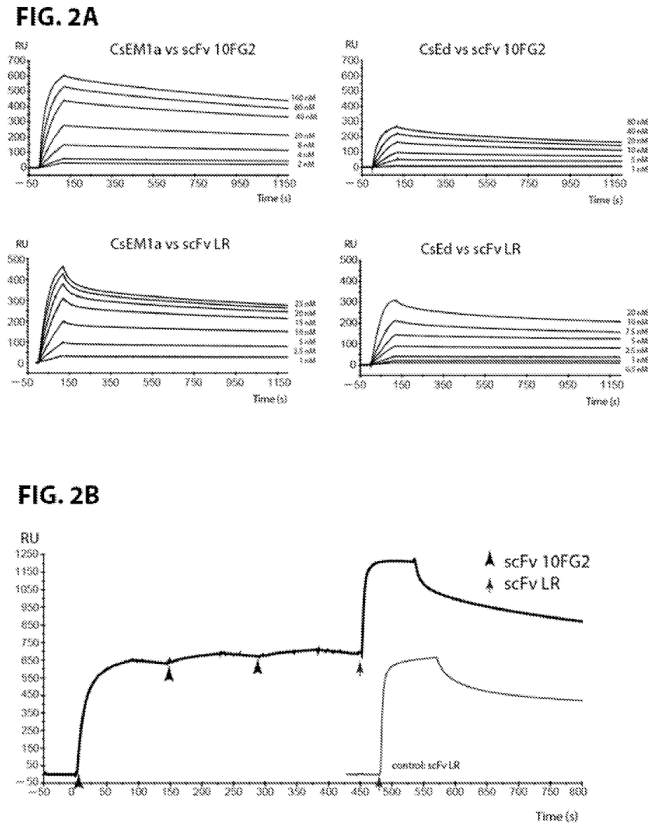

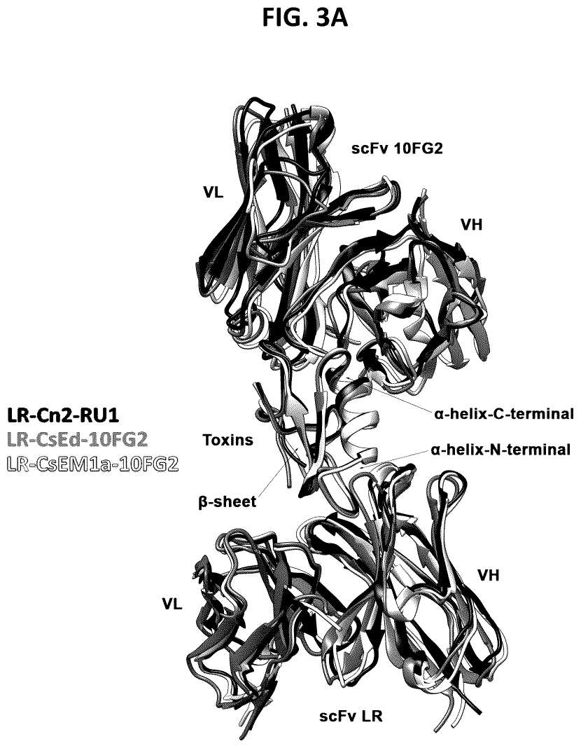

In the present disclosure, high sequence identity of the CsEd and CsEM1a toxins was observed with the Css2 and Cn2 toxins, which are neutralized by the scFvs LR (SEQ. ID. NO: 2) and 10FG2 (SEQ. ID. NO: 1) (Riano-Umbarila, Contreras-Ferrat et al. 2011, Riano-Umbarila, Gomez-Ramirez et al. 2019). Based on these results, the interaction of CsEd and CsEM1a toxins with scFvs LR (SEQ. ID. NO: 2) and 10FG2 (SEQ. ID. NO: 1) was evaluated by surface plasmon resonance (SPR) in the BiacoreX equipment (FIG. 2A). Purified toxins were immobilized on

CM5 chips and evaluations performed as described in materials and methods. The sensorgram curves confirmed that indeed CsEd and CsEM1a toxins are well recognized by both scFvs. Additionally, competition assays were performed between scFvs LR (SEQ. ID. NO: 2) and 10FG2 (SEQ. ID. NO: 1) for CsEM1a toxin. The sensorgram in FIG. 2B shows that, after saturating the scFv 10FG2 (SEQ. ID. NO: 1) binding site on the toxin, the LR (SEQ. ID. NO: 2) binding site remains available, as a similar response to the non-competitive control is observed, confirming that they recognize different sites on the toxins. The behavior was similar in competition assays with the CsEd toxin. From the sensorgrams generated at different concentrations (FIG. 2A) the kinetic constants of the interaction and affinity determination could be determined. The scFv 10FG2 (SEQ. ID. NO: 1) showed similar affinities for the two toxins with KDs of 1.1 nM and 1.8 nM, for CsEM1a and CsEd respectively. In the case of LR (SEQ. ID. NO: 2) which also recognizes the two toxins showed greater differences with KDs of 1. 29 nM and 0.61 nM, for CsEM1a and CsEd respectively.

### Example 2. Neutralization Assays of C. sculpturatus Venom

In the present disclosure after confirming the recognition of the scFvs by the toxins, preliminary tests of neutralization of C. sculpturatus venom were performed using the scFvs separately individually and together in a composition comprising both scFvs (Table 2). In the evaluation of the scFv LR (SEQ. ID. NO: 2), although there is a clear delay in the onset of intoxication signs, 90% of the population was protected with 1 LD₅₀ and 50% with 2 LD₅₀ of venom. These results contrast with the protection of scFv 10FG2 (SEQ. ID. NO: 1), which allowed the survival of the mice, but still with minimal intoxication signs using 2 LD₅₀ of venom. The scFvs used individually showed that both are able to delay the onset of intoxication signs and time to death of the animals, achieving a higher number of survivors compared to the control. It was evident that scFv 10FG2 (SEQ. ID. NO: 1) is the one that provides greater protection. What is novel and relevant is that when combining the two scFvs in a composition for the mixing assay, at a low molar ratio the neutralization was complete (Table 2).

TABLE 2
Preliminary assays of neutralization of C. sculpturatus venom

| | LD₅₀ Of | Molar ratio | |
|---|---|---|---|
| scFv | venom | Toxin:scFv(s) | Survivors/total |
| LR | 1 | 1:10 | 9/10 ** |
| LR | 2 | 1:10 | 3/6 *** |
| 10FG2 | 2 | 1:10 | 6/6 * |
| Composition LR + 10FG2 | 2 | 1:5: of each | 6/6 |
| Control | 1 | — | 5/10 *** |
| Control | 2 | — | 0/6 *** |

Tests conducted show that a composition in which scFv 10FG2 (SEQ. ID. NO: 1) is combined with scFv LR (SEQ. ID. NO: 2), protects better, as no signs of envenoming are observed even using a lower molar ratio. Signs related to the effects of the toxic components of the venom can be * minimal, such as bristly hair and itching; ** medium, involuntary tail twitching, abdominal contraction; *** strong, salivation, respiratory distress, leg paralysis, death. The molar ratio of toxins and antibodies is established considering that toxins correspond to ~10% of the venom.

In the present disclosure as a comparison criterion in the neutralization of C. sculpturatus venom, a series of tests were implemented by increasing the number of lethal doses of venom against scFv 10FG2 (SEQ. ID. NO: 1), where the same amount of scFv that neutralizes 1 LD₅₀ of venom (87 μg of scFv per mouse) was maintained. The results showed that the 87 μg of scFv 10FG2 (SEQ. ID. NO: 1) per mouse protects up to 5 LD₅₀ (Table 3A); however, intoxication signs were evident with 4 LD₅₀ and 5 LD₅₀, so it was decided to evaluate a composition comprising both scFvs 10FG2 (SEQ. ID. NO: 1) and LR (SEQ. ID. NO: 2) (Table 3A). The results show that together, in one composition, they protect and completely eliminate intoxication signs using up to 4 and 5 DL₅₀. Subsequently, the rescue test mimicking sting accidents was performed, where mice were intoxicated with 3 DL₅₀ and after 5 to 10 min a composition comprising the two scFvs was administered (Table 3B) and it was clear how the symptoms associated with scorpion sting intoxication are eliminated. After about 30 min the mice slept and ate similarly to the untreated mice, in contrast to the control which progressively developed intoxication symptoms and died in about 1 h.

TABLE 3
Formal neutralization assays of C. sculpturatus
venom using scFv 10FG2 alone and/or the composition
scFv LR + scFv 10FG2 as neutralization agents.

| | Survivors/Total | | | |
|---|---|---|---|---|
| | No. of LDs | | | |
| | Molar Ratio (toxin:scFv (s)) | | | |
| | 2 LD₅₀ | 3 LD₅₀ | 4 LD₅₀ | 5 LD₅₀ |
| | 1:5 | 1:3.3 | 1:2.5 | 1:2 |
| A. Mixing Test | | | | |
| scFv 10FG2 | 6/6 | 6/6 | 6/6 * | 6/6** |
| Composition LR + 10FG2 | — | — | 6/6 | 6/6 |
| Control | | | | |
| 2 LD₅₀ | 0/6 | | | |
| B. Rescue of mice intoxicated | | | | |
| with 3 LDs₅₀ | | | | |
| Composition scFvs LR and 10FG2 | | 6/6 | | |
| Control | | 0/6 | | |

A. Comparison of neutralization between scFv 10FG2 and composition with scFv 10FG2 plus scFv LR with 2 to 5 LD₅₀ of complete venom.
B. Rescue of intoxicated mice with 3 LD₅₀ using the composition of both scFvs at a toxin:scFvs molar ratio of 1:5 of each.

### Example 3. Structural Analysis of Complexes of scFvs 10FG2 (SEQ ID NO: 1) and LR (SEQ ID NO: 2) With CsEM1a and CsEd Toxins

In the present disclosure from the structures collected along the molecular dynamics (MD) process carried out with the toxins CsEM1a and CsEd in complex with the scFvs LR (SEQ. ID. NO: 2) and 10FG2 (SEQ. ID. NO: 1) (see methods section), the type and number of interactions present at the interface of these complexes were determined. In Tables 4 and 5, the interactions observed at the binding interface of CsEM1a and CsEd toxins with scFvs 10FG2 (SEQ. ID. NO: 1) and LR (SEQ. ID. NO: 2) are reported. FIGS. 3A to 3E show the overlay of the structural complexes of the toxins with the two antibodies, where some of the most important contacts of each toxin with the two scFv are highlighted. Similarly, the interactions of the Cn2 interface with both scFvs are compared as a reference (Riano-Umbarila, Contreras-Ferrat et al. 2011, Riano-Umbarila, Gomez-Ramirez et al. 2019) (Tables 4 and 5), since this toxin is recognized with higher affinity by the scFvs 10FG2 (SEQ. ID. NO: 1) and LR (SEQ. ID. NO: 2). Due to the similarity between the structures of the CsEM1a and CsEd toxins with the Cn2 toxin, most of the contacts at the toxin-scFvs binding interface in the molecular dynamics of the different complexes are conserved (Tables 4 and 5). These observations explain the ability of scFvs LR (SEQ. ID. NO: 2) and 10FG2 (SEQ. ID. NO: 1) to recognize this group of toxins. However, there are some differences in the way the toxins interact with the scFvs at the CDR level. Details of these differences can be seen in FIGS. 3B and 3C with 10FG2 (SEQ. ID. NO: 1) and in FIGS. 3D and 3E for LR (SEQ. ID. NO: 2).

TABLE 4
Comparison of interactions or contacts between scFv
10FG2 (SEQ. ID. NO: 1) and the indicated toxins

| | | | |
|---|---|---|---|
| | | | CsEM1a |
| 10FG2 Residue | Cn2 Residue | CsEd Residue | Residue[atom] |
| [atom] | [atom] Å^{a} | [atom] Å^{a} | Å^{a} |

Hydrogen Bonds

| | | | |
|---|---|---|---|
| S31[O] | K35 [HZ] 1.95 (9) | K35 [HZ2] 2.30 (1) | K35 [HZ2] 2.30 (1) |
| S31[OG] | K35 [HZ] 2.21 (5) | | |
| Y53[HH] | K30[O] 1.76 (10) | R30[O] 1.73 (11) | R30[O] 1.87 (10) |
| G54[H] | Q31[OE1] 2.40 (2) | Q31 [OE1] 231 (8) | Q31[OE1] 2.39 (3) |
| G55[H] | | Q31[OE1] 2.19 (1) | |
| G56[H] | Q31[OE1] 2.33 (4) | Q31[OE1] 2.36 (6) | Q31[OE1] 2.32 (3) |
| Y59[OH] | Q32 [HE22] 2.25 (4) | Q32 [HE22] 2.16 (2) | Q32 [HE22] 2.37 (1) |
| Y59[OH] | K8[H] 2.26 (3) | K8[H] 2.29 (4) | S8 [H] 2.13 (3) |
| Y59[OH] | | Y14[HH] 1.96 (1) | |
| Y59[HH] | Q32[OE1] 1.78 (6) | Q32[OE1] 1.80 (9) | Q32 [OE1] 1.99 (6) |
| Y59[HH] | D7[OD2] 2.05 (2) | | N7[OD1] 2.03 (1) |
| Y60[O] | K8 [HZ] 1.91 (2) | K8[HZ] 2.24 (2) | |
| Y60[O] | K8[HZ3] 2.04 (1) | | |
| Y60[H] | | | Y9[OH] 2.28 (6) |
| R101[HH] | | Q54 [OE1] 2.13 (2) | |
| D102[OD2] | K35[H] 1.90 (10) | K35[H] 2.05 (6) | K35[H] 2.03 (2) |
| D102[OD1] | | K35[H] 2.10 (2) | K35[H] 2.11 (3) |
| D102[OD2] | K35 [HZ] 1.77 (11) | K35 [HZ] 1.70 (5) | K35 [HZ] 1.70 (3) |
| D102[OD] | Y52[HH] 1.69 (9) | Y52[HH] 1.80 (11) | Y52[HH] 1.69 (9) |
| D102[OD1] | | K8 [HZ3] 1.79 (1) | |
| D102[OD1] | | K8[H] 1.94 (1) | |
| C103[SG] | | Q32[O] 3.90 (1) | Q32[O] 3.70 (3) |
| L104[H] | Q32[O] 1.93 (11) | Q32[O] 2.00 (11) | Q32[O] 2.07 (10) |
| L105[O] | I56[H] 2.04 (9) | V56[H] 2.16 (8) | V56[H] 2.13 (10) |
| L105[H] | Q32[O] 2.43 (3) | | Q32[O] 2.24 (5) |
| S107[OG] | Q54 [O] 3.36 (2) | Q54 [O] 3.24 (5) | Q54[O] 3.56 (7) |
| S107[OG] | Q54 [HE21] 2.04 (3) | | |
| S107[H] | Q54[O] 2.11 (11) | Q54[O] 1.94 (11) | Q54[O] 2.10 (11) |
| D108[OD] | Q54 [HE21] 2.17 (3) | Q54 [HE21] 1.93 (2) | |
| T172[OG1] | Q54 [HE22] 2.07 (4) | | |
| D233[O] | N62 [HD2] 2.00 (3) | N62 [HD21] 2.31 (3) | N62 [HD21] 2.07 (2) |
| S234[OG] | N62 [HD22] 2.06 (1) | | |
| T235[OG1] | | N62[O] 3.85 (3) | |
| T235[O] | K8[HZ3] 2.20 (3) | K8[HZ] 2.29(6) | |
| T235[O] | | K63 [HZ3] 2.12 (2) | |
| L236[O] | K8[HZ] 2.04 (5) | K8[HZ1] 2.01 (6) | |

Salt bridges

| | | | |
|---|---|---|---|
| D62[OD2] | K8 [NZ] 3.50 (4) | | |
| D102[OD2] | K35 [NZ] 2.73 (10) | K35 [NZ] 2.73 (6) | K35 [NZ] 2.67 (2) |

Hydrophobic Interactions 5Å range

| | | | |
|---|---|---|---|
| Y59 | | | Y9 |
| Y60 | | | Y9 |
| L105 | Y33 | Y33 | Y33 |
| L105 | L5 | L5 | L5 |
| L105 | V6 | | |
| L105 | I56 | V56 | V56 |
| L105 | V6 | V6 | V6 |
| W231 | I56 | | |
| L236 | I56 | | |
| L236 | L60 | L60 | L60 |
| L236 | P61 | | |

Aromatic-Aromatic Interactions from 4.5 to 7 Angstroms [Distance, angle].

| | |
|---|---|
| Y59 | Y9[5.16, 103.02] (11) |

5.3Å Aromatic-Sulfur Interactions [Distance, Angle].

| | | | |
|---|---|---|---|
| C106 | Y52[5.08, 70.31] (6) | Y52[4.57, 44.53] (8) | Y52[4.92, 55.77] (8) |
| C103 | Y52[5.11, 69.20] (2) | Y52[4.87, 52.39] (9) | Y52[5.02, 59.82] (5) |
| C103 | Y33[5.02, 98.77] (2) | Y33[5.22, 97.54] (1) | Y33[5.22, 97.66] (1) |

6Å Cation-Pi Interactions [Distance, Angle].

| | | | |
|---|---|---|---|
| R101 | Y52[4.97, 22.28] (8) | Y52[5.23, 18.68] (7) | Y52[5.29, 17.95] (10) |
| Y53 | K35[5.38, 164.59] (9) | K35[5.12, 163.28] (5) | K35[5.19, 152.31] (6) |
| Y32 | K35[5.93, 37.01] (2) | | |
| W47 | K8[5.87, 86.41] (3) | K8[5.88, 76.83] (3) | |
| Y59 | K8[5.35, 27.64] (4) | K8[5.50, 22.82] (3) | |
| K65 | | | Y9[4.78, 23.00] (10) |
| W231 | K8[5.77, 70.82] (1) | K8[5.77, 70.79] (1) | |

^{a}Average distance in different frames of the MD sample. The number in parentheses indicates how many times the contact was observed in the MD sample. Bold highlights indicate interactions with the main chain of the indicated residue.

TABLE 5
Comparison of interactions or contacts between
scFv LR (SEQ. ID. NO: 2) and the listed toxins

| | | | |
|---|---|---|---|
| LR Residue | Cn2 Residue | CsEd Residue | CsEM1a Residue |
| [atom] | [atom] Å^{a} | [atom] Å^{a} | [atom] Å^{a} |

Hydrogen Bonds

| | | | |
|---|---|---|---|
| N31[O] | | E15[H] 2.15 (15) | |
| N31[HD2X] | | K13[O] 2.05 (17) | |
| N31[OD1] | | N10 [HD2X] 1.97 (21) | |
| N31[HD22] | | N10[OD1] 2.17 (3) | |
| Y32[HH] | S66[O] 1.79 (4) | S66[O] 1.81 (1) | N66[O] 1.78 (1) |
| Y32[HH] | S66[OG] 1.77 (1) | | |
| Y32[OH] | C65 [SG] 3.41 (1) | C65 [SG] 3.64 (3) | |
| Y32[HH] | C65[O] 2.06 (2) | | |
| Y32[OH] | | C12[SG] 3.46 (3) | |
| A33[H] | | E15 [OEX] 1.86 (19) | |
| H35[NE2] | E15[OE2] 3.66 (1) | E15[OE2] 3.66 (2) | E15[OE2] 3.68 (4) |
| R53[HH1X] | D7[ODX] 1.85 (24) | N7[OD1] 1.91 (4) | N7[OD1] 2.20 (2) |
| R5 3[HH11] | K13[O] 2.05 (15) | | |
| R53[O] | Y24[HH] 1.65 (3) | Y24[HH] 1.88 (1) | Y24[HH] 1.64 (1) |
| R53[HH12] | C12 [O] 2.13 (1) | | |
| R53[HH22] | | E28[OE2] 1.83 (1) | |
| R53[HH21] | | Y14[OH] 1.98 (2) | |
| R53[HH12] | | | Y14[OH] 2.05 (1) |
| R53[O] | | R27 [HH22] 2.27 (3) | |
| S54[OG] | R27 [HH22] 2.18 (6) | | |
| S54[O] | R27 [HH22] 1.95 (3) | R27 [HH22] 2.02 (11) | |
| S55[O] | R27 [HHX2] 1.99 (4) | R27[HHX2] 1.79 (5) | |
| G56[O] | | | R27 [HH22] 2.33 (3) |
| D57[ODX] | L17[H] 1.84 (21) | L17[H] 1.83 (20) | L17[H] 1.89 (19) |
| D57[ODX] | K18[H] 2.03 (19) | K18[H] 2.10 (19) | K18[H] 1.99 (18) |
| D57[OD1] | K18 [HZX] 1.71 (4) | K18 [HZX] 1.71 (16) | K18 [HZX] 1.71 (19) |
| D57[OD1] | | K18 [NZ] 2.67 (1) | |
| D57[ODX] | N22 [HD22] 2.00 (19) | N22 [HD22] 1.91 (15) | N22 [HD22] 1.88 (14) |
| D57[O] | | | Y24[HH] 1.61 (1) |
| I58[O] | K18 [HZX] 1.91 (2) | K18 [HZX] 1.81 (14) | K18 [HZX] 1.94, (12) |
| D59[OD2] | K18 [HZ3] 1.69 (1) | | |
| D59[OD2] | K18 [HZ1] 1.60 (1) | K18 [HZ1] 2.03 (1) | |
| R98[HH22] | S66[O] 2.41 (1) | | |
| G100[H] | E15 [OE2] 2.17 (1) | E15 [OEX] 2.15 (8) | E15 [OEX] 2.16, (8) |
| F101[H] | E15[OE1] 1.18 (12) | E15 [OEX] 1.99 (9) | E15 [OEX] 2.01 (14) |
| G102[H] | E15 [OEX] 2.05 (14) | E15 [OEX] 2.11 (10) | E15 [OE2] 2.14 (13) |
| R163[HH22] | Y42[OH] 1.78 (1) | | |
| R163[HH11] | Y42[OH1] 2.10 (1) | | |
| Y165[HH] | Y4[OH] 2.32 (1) | | Y4[OH] 2.09 (4) |
| Y165[OH] | Y42[HH] 2.07 (1) | | |
| R229[HHX2] | E15 [OEX] 1.88 (34) | E15 [OEX] 1.96 (38) | E15 [OEX] 1.87 (31) |
| R229[HH21] | C16[O] 2.28 (2) | C16[O] 2.33 (1) | C16[O] 2.50 (1) |

Salt bridges

| | | | |
|---|---|---|---|
| H35[NE2] | E15 [OEX] 3.78 (4) | E15 [OEX] 3.66 (2) | E15 [OEX] 3.74 (9) |
| R53[ ]NH1] | D7[OD2] 3.28 (11) | | |
| R53[ ]NH1] | D7[OD1] 3.87 (5) | | |
| R53[NH2] | D7[OD2] 2.79 (13) | | |
| R53[NH2] | D7[OD1] 3.18 (10) | | |
| R53[ ]NH1] | | E28[OE2] 2.81 (1) | |
| R53[ ]NH2] | | E28[OE2] 2.81 (1) | |
| D57[ODX] | K18 [NZ] 2.73 (7) | K18 [NZ] 2.81 (18) | K18 [NZ] 2.71 (19) |
| R98[NHX] | S66[O] 3.23 (1) | S66[O] 3.79 (1) | N66[O] 3.58 (2) |
| R229[NHX]. | E15 [OEX] 3.16 (39) | E15[OE2] 2.92 (39) | E15[OE2] 2.81 (37) |

Hydrophobic interactions within 5Å

| | | | |
|---|---|---|---|
| A33 | L17 (9) | L17 (18) | L17 (10) |
| W47 | L17 (4) | L17 (11) | L17 (12) |
| I51 | | | L17 1 |
| F101 | A45 (2) | A45 (19) | A45 (13) |
| F101 | | F44 (8) | |
| F101 | | | W58 (1) |
| Y165 | Y4 (1) | | Y4 (4) |
| Y165 | Y42 (14) | Y42 (3) | Y42 (11) |
| Y165 | A43 (19) | A43 (13) | A43 (19) |
| Y224 | F44 (20) | F44 (20) | F44 (18) |
| Y224 | A43 (4) | A43 (2) | A43 (3) |
| Y226 | L19 (2) | L19 (1) | L19 (2) |

Aromatic-Aromatic Interactions from 4.5 to 7 Angstroms [Distance, Angle].

| | | | |
|---|---|---|---|
| W47 | | F44 [6.80; 100.67] (1) | F44 [6.74; 126.53] (1) |
| F101 | | W58 [6.97; 109.02](1) | W58 [6.05; 100.86] (1) |
| Y165 | Y42[6.47; 86.29] (13) | Y42 [6.27; 87.96] (4) | Y42 [6.47; 84.29](13) |
| Y65 | | | Y4[6.82; 80.36] (1) |
| Y224 | F44[6.77; 105.36] (6) | F44[6.93; 110.57] (1)] | F44 [6.87; 119.96] (2) |

5.3Å Aromatic-Sulfur Interactions [Distance, Angle].

| | | | |
|---|---|---|---|
| Y32 | C12[5.01; 32.08] (3) | C12[5.16; 56.54] (1) | |
| Y32 | C65[4.74; 48.75] (17) | C65[4.80; 50.80] (1) | C65 [4.55; 61.04] (1) |
| F101 | C65[5.28; 72.12] (17) | C65[4.52; 90.00] (2) | C65 [4.62; 70.78] (7) |
| F101 | C12[5.04; 56.73] (1) | | C12 [4.92; 57.80] (2) |

6Å Cation-Pi Interactions [Distance, Angle].

| | | | |
|---|---|---|---|
| R53 | Y14[3.92; 17.28] (20) | Y14 [5.23; 126.92] (17) | Y14 [5.03; 135.42](9) |
| F101 | K13[5.55; 53.05] (7) | K13 [5.08; 33.29] (18) | K13 [5.41; 46.96] (8) |
| Y165 | | | K13[5.86; 90.18] (1) |
| R225 | | Y42 [5.51; 99,76] (3) | Y42 [5.15; 71.17](5) |
| R229 | F44[4.58; 40.18] (19) | F44 [4.50; 28.30] (20) | F44 [4.34; 30.66](19) |

^{a}Average distance in different “frames” of the sample taken from the MDs. The number in parentheses indicates how many times the contact was observed in the MDsample. Bold letters indicate interactions with the main chain of the indicated residue.

Temperature factors B of the first 20 residues of
CsEM1a and CsEd toxins andtheir differences during
interaction with scFv 10FG2 (SEQ. ID. NO: 1).

| | | CsEM1a | | CsEd | Difference |
|---|---|---|---|---|---|
| Waste | | B factor | | B factor | CsEM1a − CsEd |
| 1 | K | 167.6296969387 | K | 35.302643674 | 132.3270532647 |
| | E | 18.054839999 | E | 19.262340288 | −1.207500289 |
| | G | 5.0506361493 | G | 4.4643463912 | 0.5862897581 |
| | Y | 5.9322012627 | Y | 5.2696473751 | 0.6625538876 |
| HH 5 | L | 19.9735798205 | L | 16.7065914745 | 3.2669883459 |
| HH 6 | V | 14.8477939047 | V | 15.6693531253 | −0.8215592207 |
| C 7 | N | 36.0077170784 | N | 39.8229365656 | −3.8152194872 |
| CC 8 | S | 32.8495186243 | K | 40.3840991739 | −7.5345805496 |
| C 9 | Y | 25.3281339078 | S | 49.2956494218 | −23.9675155141 |
| | T | 19.321280032 | N | 34.4138114961 | −15.0925314641 |
| | G | 8.2076367038 | G | 10.9841040141 | −2.7764673103 |
| | C | 7.7108895193 | C | 10.4611618013 | −2.750272282 |
| | K | 35.2774719618 | K | 38.9140703605 | −3.6365983987 |
| C 14 | Y | 57.3110461853 | Y | 49.485018431 | 7.8260277543 |
| | E | 38.5993317058 | E | 35.0650328579 | 3.5342988479 |
| | C | 5.9607549183 | C | 5.1599951324 | 0.8007597859 |
| | L | 50.2558412015 | L | 53.5181320732 | −3.2622908717 |
| | K | 73.2126242526 | K | 80.8775249039 | −7.6649006513 |
| | L | 25.1397119297 | L | 20.7111697703 | 4.4285421594 |
| | G | 8.0152927149 | G | 6.8558417737 | 1.1594509412 |

### REFERENCES

Boyer, L., J. Degan, A. M. Ruha, J. Mallie, E. Mangin and A. Alagon (2013). “Safety of intravenous equine F(ab′)2: insights following clinical trials involving 1534recipients of scorpion antivenom.” Toxicon 76: 386-393.

Carcamo-Noriega, E. N., T. Olamendi-Portugal, R. Restano-Cassulini, A. Rowe, S. J. Uribe-Romero, B. Becerril and L. D. Possani (2018). “Intraspecific variation of Centruroides sculpturatus scorpion venom from two regions of Arizona.” Arch Biochem Biophys 638: 52-57.

Gomez-Ramirez, I. V., L. Riano-Umbarila, T. Olamendi-Portugal, R. Restano-Cassulini, L. D. Possani and B. Becerril (2020). “Biochemical, electrophysiological and immunological characterization of the venom from Centruroides baergi, a new scorpion species of medical importance in Mexico.” Toxicon 184: 10-18.

Gonzalez-Santillan, E. and L. D. Possani (2018). “North American scorpion species of public health importance with a reappraisal of historical epidemiology.” Acta Trop 187: 264-274.

Gummin, D. D. D., J. B. Mowry, M. C. Beuhler, D. A. Spyker, D. E. Brooks, K. W. Dibert, L. J. Rivers, N. P. T. Pham and M. L. Ryan (2020). “2019 Annual Report of the American Association of Poison Control Centers' National Poison Data System (NPDS): 37th Annual Report.” Clin Toxicol (Phila) 58(12): 1360-1541.

Kang, A. M. and D. E. Brooks (2017). “Geographic Distribution of Scorpion Exposures in the United States, 2010-2015.” Am J Public Health 107(12): 1958-1963.

Kevin J. Bowers, E. C., Huafeng Xu, Ron O. Dror, Michael P. Eastwood, J. L. K. Brent
A. Gregersen, Istvan Kolossvary, Mark A. Moraes, Federico D. Sacerdoti, and Y.
S. John K. Salmon, David E. Shaw (2006). S. John K. Salmon, David E. Shaw (2006). “Scalable algorithms for molecular dynamics simulations on commodity clusters.” Proceeding (84): 1-13.

Knudsen, C., N. R. Casewell, B. Lomonte, J. M. Gutierrez, S. Vaiyapuri and A. H. Laustsen (2020). “Novel Snakebite Therapeutics Must Be Tested in Appropriate Rescue Models to Robustly Assess Their Preclinical Efficacy.” Toxins (Basel) 12(9).

Krissinel, E. and K. Henrick (2007). “Inference of macromolecular assemblies from crystalline state.” J Mol Biol 372(3): 774-797.

Lopez-Giraldo, A. E., T. Olamendi-Portugal, L. Riano-Umbarila, B. Becerril, L. D. Possani, M. Delepierre and F. Del Rio-Portilla (2020). “The three-dimensional structure of the toxic peptide CI13 from the scorpion Centruroides limpidus.” Toxicon 184: 158-166.

Olamendi-Portugal, T., R. Restano-Cassulini, L. Riano-Umbarila, B. Becerril and L. D. Possani (2017). “Functional and immuno-reactive characterization of a previously undescribed peptide from the venom of the scorpion Centruroides limpidus.” Peptides 87: 34-40.

Pucca, M. B., F. A. Cerni, R. Janke, E. Bermudez-Mendez, L. Ledsgaard, J. E. Barbosa and A. H. Laustsen (2019). “History of Envenoming Therapy and Current Perspectives.” Front Immunol 10: 1598.

Riano-Umbarila, L., G. Contreras-Ferrat, T. Olamendi-Portugal, C. Morelos-Juarez, G. Corzo, L. D. Possani and B. Becerril (2011). “Exploiting cross-reactivity to neutralize two different scorpion venoms with one single chain antibody fragment.” J Biol Chem 286(8): 6143-6151.

Riano-Umbarila, L., I. V. Gomez-Ramirez, L. M. Ledezma-Candanoza, T. Olamendi-Portugal, E. R. Rodriguez-Rodriguez, G. Fernandez-Taboada, L. D. Possani and B. Becerril (2019). “Generation of a Broadly Cross-Neutralizing Antibody Fragment against Several Mexican Scorpion Venoms.” Toxins (Basel) 11(1).

Riano-Umbarila, L., V. R. Juarez-Gonzalez, T. Olamendi-Portugal, M. Ortiz-Leon, L. D. Possani and B. Becerril (2005). “A strategy for the generation of specific human antibodies by directed evolution and phage display. An example of a single-chain antibody fragment that neutralizes a major component of scorpion venom.” Febs J 272(10): 2591-2601.

Riano-Umbarila, L., L. M. Ledezma-Candanoza, H. Serrano-Posada, G. Fernandez- Taboada, T. Olamendi-Portugal, S. Rojas-Trejo, I. V. Gomez-Ramirez, E. Rudino-Pinera, L. D. Possani and B. Becerril (2016). “Optimal Neutralization of Centruroides noxius Venom Is Understood through a Structural Complex between Two Antibody Fragments and the Cn2 Toxin.” The Journal of biological chemistry 291(4): 1619-1630.

Riano-Umbarila, L., E. R. Rodriguez-Rodriguez, C. E. Santibanez-Lopez, L. Guereca, S. J. Uribe-Romero, I. V. Gomez-Ramirez, E. N. Carcamo-Noriega, L. D. Possani and B. Becerril (2017). “Updating knowledge on new medically important scorpion species in Mexico.” Toxicon 138: 130-137.

Riano-Umbarila, L., V. M. Rojas-Trejo, J. A. Romero-Moreno, M. Costas, I. Utrera-Espindola, T. Olamendi-Portugal, L. D. Possani and B. Becerril (2020). “Comparative assessment of the VH-VL and VL-VH orientations of single-chain variable fragments of scorpion toxin-neutralizing antibodies.” Mol Immunol 122: 141-147.

Röthlisberger D, Honegger A, Plückthun A. Domain interactions in the Fab fragment: a comparative evaluation of the single-chain Fv and Fab format engineered with variable domains of different stability. J Mol Biol. 2005 Apr. 8; 347(4): 773-89. doi: 10.1016/j.jmb.2005.01.053. PMID: 15769469.

Schiavon, E., M. Pedraza-Escalona, G. B. Gurrola, T. Olamendi-Portugal, G. Corzo, E. Wanke and L. D. Possani (2012). “Negative-shift activation, current reduction and resurgent currents induced by beta-toxins from Centruroides scorpions in sodium channels.” Toxicon 59(2): 283-293.

Tina, K. G., R. Bhadra and N. Srinivasan (2007). “PIC: Protein Interactions Calculator.”Nucleic Acids Res 35(Web Server issue): W473-476.

1. A composition that neutralizes Centruroides sculpturatus scorpion venom, comprising an antibody fragment 10FG2, and an antibody fragment LR.

2. The composition according to claim 1, wherein the antibody fragments 10FG2, and LR, are in scFv format, having sequences SEQ. ID. NO: 1 and SEQ.ID. NO:2, respectively.

3. The composition according to claim 1, wherein the antibody fragments 10FG2, and LR, are in Fab format, having amino acidic sequences SEQ. ID. NO: 3 for the light chain and SEQ.ID. No:4 for the heavy chain, as well as SEQ. ID. NO: 5 for the light chain and SEQ.ID. NO:6 for the heavy chain, respectively.

4. The composition of claim 1 wherein the composition completely neutralize Centruroides sculpturatus scorpion venom.

5. A pharmaceutical composition that neutralizes Centruroides sculpturatus scorpion venom, comprising an antibody fragment 10FG2, and an antibody fragment LR and a pharmaceutically acceptable vehicle.

6. The pharmaceutical composition of claim 5, wherein the antibody fragments 10FG2, and LR, are in scFv format, having amino acidic sequences SEQ. ID. NO: 1 and SEQ. ID. NO: 2, respectively.

7. The pharmaceutical composition according to claim 5, wherein the antibody fragments 10FG2, and LR, are in Fab format, having amino acidic sequences SEQ. ID. NO: 3 for the light chain and SEQ.ID. No:4 for the heavy chain, as well as SEQ. ID. NO: 5 for the light chain and SEQ.ID. NO:6 for the heavy chain, respectively.

8. The pharmaceutical composition of claim 5 wherein the pharmaceutical composition completely neutralizes Centruroides sculpturatus scorpion venom.

9. A method for treating Centruroides sculpturatus scorpion envenoming comprising the step of administering the pharmaceutical composition of claim 5 to neutralize the Centruroides sculpturatus scorpion venom.

10. A method for treating Centruroides sculpturatus scorpion envenoming comprising the step of administering the pharmaceutical composition of claim 6 to neutralize the Centruroides sculpturatus scorpion venom.

11. A method for treating Centruroides sculpturatus scorpion envenoming comprising the step of administering the pharmaceutical composition of claim 7 to neutralize the Centruroides sculpturatus scorpion venom.


SEQUENCE LISTING (6 sequences)

SEQ ID NO: 1 - protein, 273 aa, synthetic construct
  REGION 1..273: note = Matured Antibody fragment
  REGION 1..273: note = mat_peptide - scFv 10FG2
QVNLRESGGGLVQPGGSLRLSCAASGFSFGSYGMHWVRQAPGKGLEWVAVISYGGGSKYYADSVKGRFTISRDNSKNTLYLQMNSLRAEDTAVYYCAKDARDCLLCSDWTFDLWGRGTLVTVSSGGGGSGGGGSGGGGSNFMLTQPHSASGTPGQRVTISCSGSSSNIGSNTVNWYRHLPGSAPELLIGSHNQRPSGVPDRFSGSKSGTSASLAISGLQSEDEADYYCAAWDDSTLGYVFGTGTKLTVLGAAAEQKLISEEDLNGAAHHHHHH

SEQ ID NO: 2 - protein, 263 aa, synthetic construct
  REGION 1..263: note = Matured antibody fragment
  REGION 1..263: note = mat_peptide - acFv LR
EVQLVESGGGLVQPGGSLRLSCTGSGFTFDNYAMHWLRQVPGEGLEWVSGISRSSGDIDYADSVKGRFTISRDDAKKTLSLQMNSLRAEDTAVYYCARGGFGSFDTWGQGTMVTVSSGGGGSGGGGSGGGGSEIVLTQSPATLSVSPGERATLSCRASQSVRSYLAWYQQKPGQAPRLLFSDASNRATGIPARFTGSGSGTDFTLTISSLEPEDFAIYYCQQYRYSPRTFGQGTKVEIKRAAAEQKLISEEDLNGAAHHHHHH

SEQ ID NO: 3 - protein, 218 aa, synthetic construct
  REGION 1..218: note = Fab 10FG2 Light Chain (VL-CL)
  REGION 1..218: note = mat_peptide - Fab 10FG2 Light Chain (VL-CL)
NFMLTQPHSASGTPGQRVTISCSGSSSNIGSNTVNWYRHLPGSAPELLIGSHNQRPSGVPDRFSGSKSGTSASLAISGLQSEDEADYYCAAWDDSTLGYVFGTGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGEC

SEQ ID NO: 4 - protein, 250 aa, synthetic construct
  REGION 1..250: note = Fab 10FG2 Heavy Chain (VH-CH)
  REGION 1..250: note = mat_peptide - Fab 10FG2 Heavy Chain (VH-CH)
QVNLRESGGGLVQPGGSLRLSCAASGFSFGSYGMHWVRQAPGKGLEWVAVISYGGGSKYYADSVKGRFTISRDNSKNTLYLQMNSLRAEDTAVYYCAKDARDCLLCSDWTFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAEQKLISEEDLNGAAHHHHHH

SEQ ID NO: 5 - protein, 214 aa, synthetic construct
  REGION 1..214: note = Fab LR Heavy Chain (VL-CL)
  REGION 1..214: note = mat_peptide - Fab LR Light Chain (VL-CL)
EIVLTQSPATLSVSPGERATLSCRASQSVRSYLAWYQQKPGQAPRLLFSDASNRATGIPARFTGSGSGTDFTLTISSLEPEDFAIYYCQQYRYSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGEC

SEQ ID NO: 6 - protein, 243 aa, synthetic construct
  REGION 1..243: note = Fab LR Heavy Chain (VH-CH)
  REGION 1..243: note = mat_peptide - Fab LR Heavy Chain (VH-CH)
EVQLVESGGGLVQPGGSLRLSCTGSGFTFDNYAMHWLRQVPGEGLEWVSGISRSSGDIDYADSVKGRFTISRDDAKKTLSLQMNSLRAEDTAVYYCARGGFGSFDTWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAEQKLISEEDLNGAAHHHHHH